(19) United States
(12) Patent Application Publication
(10) Pub. No.: US 2017/0265818 A1
(43) Pub. Date: Sep. 21, 2017

(54) DIAGNOSIS SUPPORT APPARATUS, OPERATING METHOD, AND NON-TRANSITORY COMPUTER READABLE MEDIUM

(71) Applicant: FUJIFILM Corporation, Tokyo (JP)

(72) Inventor: Shoji KANADA, Tokyo (JP)

(73) Assignee: FUJIFILM Corporation, Tokyo (JP)

(21) Appl. No.: 15/447,871

(22) Filed: Mar. 2, 2017

(30) Foreign Application Priority Data
Mar. 17, 2016 (JP) 2016-053292

(51) Int. Cl. A61B 5/00; A61B 5/044; A61B 5/04; A61B 5/0205

(52) U.S. Cl. CPC A61B 5/7278; A61B 5/0205; A61B 5/044; A61B 5/7405; A61B 5/04012; A61B 5/021

(57) ABSTRACT

A diagnosis support apparatus for physiological monitoring includes a data acquisition device for obtaining information of a heartbeat waveform of an examinee. An extractor extracts blood pressure fluctuations based on changes of blood pressure of the examinee from the heartbeat waveform. A guide information output device outputs guide information for induction of respiratory cycle time of breathing of the examinee according to a period of the blood pressure fluctuations. An arithmetic processor obtains a parameter value related to intensity of the heartbeat waveform. A determiner determines occurrence or non-occurrence of a problem in autonomic activity in an autonomic nervous system in the examinee according to the parameter value. A result output device outputs a determination result of the determiner. Also, the determiner performs the determining according to a maximum of the parameter value within a predetermined first period.

### CROSS-REFERENCE TO RELATED APPLICATION

This application claims priority under 35 USC 119 from Japanese Patent Application No. 2016-053292, filed 17 Mar. 2016, the disclosure of which is incorporated by reference herein.

### BACKGROUND OF THE INVENTION

1. Field of the Invention

The present invention relates to a diagnosis support apparatus, operating method, and non-transitory computer readable medium. More particularly, the present invention relates to a diagnosis support apparatus in which an examinee of a diagnostic test can be reliably kept in a relaxed state for correctly checking occurrence or non-occurrence of a problem in autonomic activity, and an operating method and non-transitory computer readable medium.

2. Description Related to the Prior Art

A heartbeat waveform is information of changes of heartbeat intervals of a heartbeat with time. Various components relevant to an autonomic nervous system are included in the heartbeat waveform, such as blood pressure fluctuations based on changes in blood pressure (medically referred to as Mayer waves), and respiratory fluctuations based on changes in breathing. It is known that occurrence of a problem in autonomic activity (physiological changes) of an examinee or patient is detected by evaluating the heartbeat waveform.

JP-B 5626853 discloses a method of detecting dementia of a patient as an examinee corresponding to either one of dementia of the Alzheimer type and dementia of Lewy body type by consideration of the heartbeat waveform. The feature of this document is focused on the clinical knowledge in that the problem in autonomic activity occurs with the dementia of Lewy body type in contrast with non-occurrence of the problem in autonomic activity in the dementia of the Alzheimer type, examples of symptoms of the problem in autonomic activity including unstable bleed pressure, a problem in adjusting the respiration, and the like. To this end, a low frequency band component (LF) corresponding to the blood pressure fluctuations is extracted from the heartbeat waveform of the examinee. A high frequency band component (HF) corresponding to the respiratory fluctuations is extracted from the heartbeat waveform of the examinee. A ratio LF/HF between those components is compared with a predetermined threshold. It is detected that the disease of the examinee is dementia of the Alzheimer type without occurrence of the problem in autonomic activity assuming that the ratio is higher than the threshold, and is dementia of Lewy body type with occurrence of the problem in autonomic activity assuming that the ratio is lower than the threshold.

The autonomic nervous system is constituted by sympathetic nerves and parasympathetic nerves. Fluctuations of the blood pressure with relatively low frequency become components of the heartbeat waveform with effect of the sympathetic nerves and the parasympathetic nerves. In contrast, fluctuations of the respiration with relatively high frequency become components of the heartbeat waveform with effect of only the parasympathetic nerves. Assuming that stress occurs in a body of the examinee, the sympathetic nerves are activated. Assuming that the body of the examinee is relaxed, the parasympathetic nerves are activated. Thus, there occurs a difference in the form of the heartbeat waveform owing to the stress or relaxation of the examinee during the diagnostic test. It is also known to perform detection as to which of the stressed state or relaxed state the examinee is in by utilization of the knowledge of the sympathetic nerves and the parasympathetic nerves.

In general, a period of the blood pressure fluctuations is as long as 10 seconds. It is medically known that the examinee can be relaxed while the period of the blood pressure fluctuations is equal to respiratory cycle time.

In performing a diagnostic test of the problem in autonomic activity according to JP-B 5626853, stress of the examinee causes noise in detecting the problem in autonomic activity by unwanted influence to the heartbeat waveform. It is necessary to keep the examinee relaxed to regularize the condition of the measurement in the diagnostic test. To this end, guide information is utilized for inducing the examinee to breathe with a respiration cycle equal to a predetermined period stored previously.

However, there is specificity in the period of the blood pressure fluctuations. Assuming that the specificity is neglected, there occurs a problem in failure in relaxing the body of the examinee in case his or her respiration is induced for respiration cycle with equality to the predetermined period as a constant value.

### SUMMARY OF THE INVENTION

In view of the foregoing problems, an object of the present invention is to provide a diagnosis support apparatus in which an examinee of a diagnostic test can be reliably kept in a relaxed state for correctly checking occurrence or non-occurrence of a problem in autonomic activity, and an operating method and non-transitory computer readable medium.

In order to achieve the above and other objects and advantages of this invention, a diagnosis support apparatus includes a data acquisition device for obtaining information of a heartbeat waveform of an examinee. An extractor extracts blood pressure fluctuations based on changes of blood pressure of the examinee from the heartbeat waveform. A guide information output device outputs guide information for induction of respiratory cycle time of breathing of the examinee according to a period of the blood pressure fluctuations. An arithmetic processor obtains a parameter value related to intensity of the heartbeat waveform. A determiner determines occurrence or non-occurrence of a problem in autonomic activity in an autonomic nervous system in the examinee according to the parameter value. A result output device outputs a determination result of the determiner.

Preferably, the determiner performs the determining according to a maximum of the parameter value within a predetermined first period.

Preferably, furthermore, a detector detects whether the period of the blood pressure fluctuations corresponds to the respiratory cycle time of the examinee.

Preferably, the detector performs detection according to a signal from a respiration sensor for detecting breathing of the examinee.

Preferably, the extractor further extracts respiratory fluctuations based on changes in breathing of the examinee from the heartbeat waveform. The detector performs detection according to the respiratory fluctuations.

Preferably, the determiner performs the determining in case a detection result of correspondence between the period of the blood pressure fluctuations and the respiratory cycle time of the examinee is continuously output by the detector for a predetermined second period.

Preferably, the guide information output device outputs the guide information in a visual form and/or audio form.

Preferably, the determiner performs the determining by comparison with the parameter value with a predetermined threshold.

In a preferred embodiment, the determiner performs the determining according to a discriminant equation in which a variant is the parameter value and which is obtained statistically according to past cases.

Preferably, the arithmetic processor obtains a power value of a low frequency band component of the heartbeat waveform for the parameter value.

Also, an operating method for a diagnosis support apparatus includes a step of obtaining information of a heartbeat waveform of an examinee. Blood pressure fluctuations based on changes of blood pressure of the examinee are extracted from the heartbeat waveform. Guide information is output for induction of respiratory cycle time of breathing of the examinee according to a period of the blood pressure fluctuations. A parameter value related to intensity of the heartbeat waveform is obtained. Occurrence or non-occurrence of a problem in autonomic activity in an autonomic nervous system in the examinee is determined according to the parameter value. A determination result of the determining step is output.

Also, a non-transitory computer readable medium for storing a computer-executable program enabling execution of computer instructions to perform operations for diagnosis support is provided. The operations include obtaining information of a heartbeat waveform of an examinee. The operations include extracting blood pressure fluctuations based on changes of blood pressure of the examinee from the heartbeat waveform. The operations include outputting guide information for induction of respiratory cycle time of breathing of the examinee according to a period of the blood pressure fluctuations. The operations include obtaining a parameter value related to intensity of the heartbeat waveform. The operations include determining occurrence or non-occurrence of a problem in autonomic activity in an autonomic nervous system in the examinee according to the parameter value. The operations include outputting a determination result of the determining.

Consequently, an examinee of a diagnostic test can be reliably kept in a relaxed state for correctly checking occurrence or non-occurrence of a problem in autonomic activity, because guide information is utilized for the examinee to induce his or her respiratory cycle time according to the period of the blood pressure fluctuations.

### BRIEF DESCRIPTION OF THE DRAWINGS

The above objects and advantages of the present invention will become more apparent from the following detailed description when read in connection with the accompanying drawings, in which:

FIG. 1 is an explanatory view in a perspective illustrating diagnosis with a diagnosis support apparatus;

FIG. 2 is a block diagram schematically illustrating a computer for constituting the diagnosis support apparatus;

FIG. 3 is a block diagram schematically illustrating circuit devices in a CPU of the diagnosis support apparatus;

FIG. 4 is a graph illustrating a data acquisition device for forming a heartbeat waveform;

FIG. 5 is an explanatory view in a graph illustrating an extraction function of an extractor;

FIG. 6 is an explanatory view in a graph illustrating an output function of an output device;

FIG. 7 is an explanatory view in a perspective illustrating outputs of the guide information;

FIG. 8 is an explanatory view in a perspective illustrating outputs of the guide information of a second form;

FIG. 9 is an explanatory view in a graph illustrating an obtaining function of an arithmetic processor;

FIG. 10 is a block diagram schematically illustrating a detecting function of a detector and an output function of the output device;

FIG. 11 is a screen view illustrating a result page for a result without a problem in an autonomic activity;

FIG. 12 is a screen view illustrating a result page for a result with a problem in the autonomic activity;

FIG. 13 is a timing chart illustrating steps of operation of circuit devices in the CPU;

FIG. 14 is a flow chart illustrating steps of operation of the diagnosis support apparatus;

FIG. 15 is a block diagram schematically illustrating a second preferred diagnosis support apparatus with circuit devices;

FIG. 16 is a block diagram schematically illustrating a detector;

FIG. 17 is a timing chart illustrating steps of determination in a determiner;

FIG. 18 is a block diagram schematically illustrating a third preferred diagnosis support apparatus with circuit devices;

FIG. 19 is an explanatory view in a graph illustrating a detector;

FIG. 20 is a flow chart illustrating steps of determination in a determiner in a fourth preferred diagnosis support apparatus;

FIG. 21 is a block diagram schematically illustrating still another preferred diagnosis support apparatus.

### DETAILED DESCRIPTION OF THE PREFERRED EMBODIMENT (S) OF THE PRESENT INVENTION

### First Embodiment

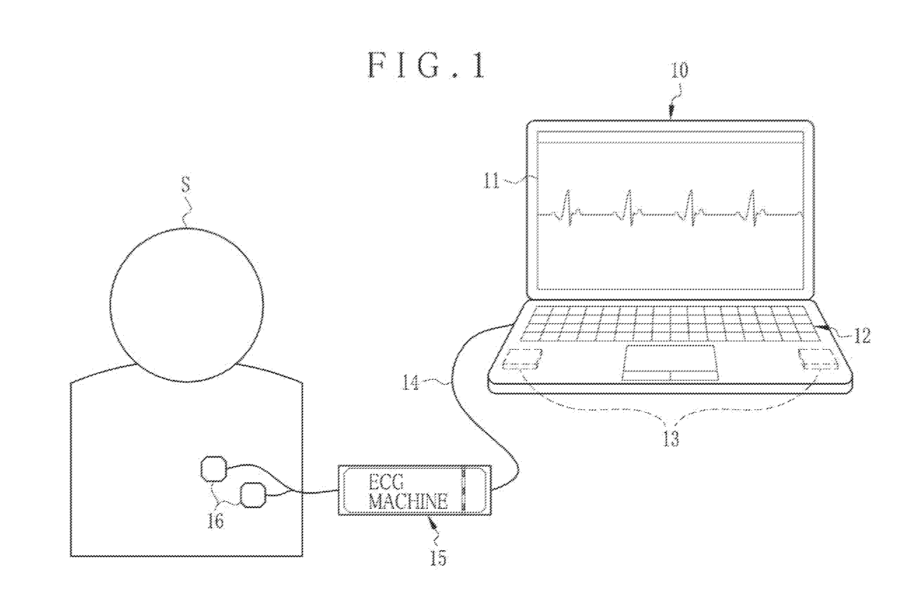

In FIG. 1, a diagnosis support apparatus 10, as medical electronic equipment in combination with a physiological monitoring apparatus, is a personal computer of a notebook type, and includes a display panel 11, a user input interface 12 or input panel, and audio speakers 13. The user input interface 12 is constituted by a keyboard, touch pad and the like.

The diagnosis support apparatus 10 is installed in a medical facility, for example, hospital, and used for supporting diagnosis of health of an examinee S. The diagnosis support apparatus 10 acquires information of a heartbeat waveform of the examinee S, checks occurrence of a problem in autonomic activity (physiological changes) in an autonomic nervous system in the examinee S according to intensity of the heartbeat waveform (intensity level of signal intensity), and outputs a result of the check.

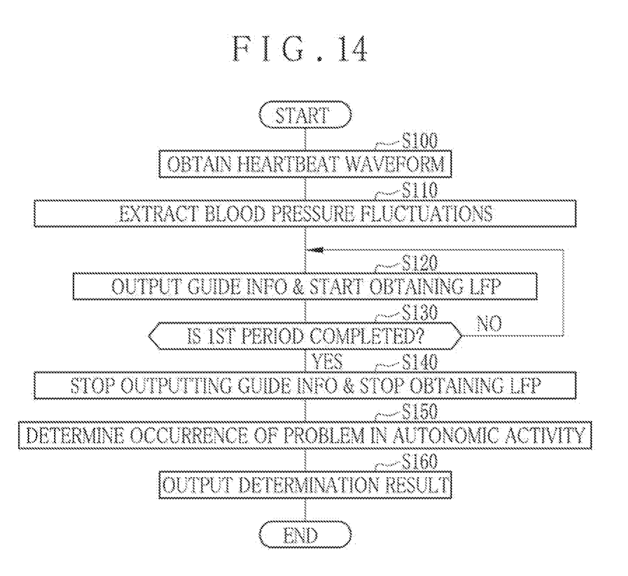

An ECG machine 15 (electrocardiogram machine) is connected to the diagnosis support apparatus 10 by use of a USB cable 14 (Universal Serial Bus cable). Electrode pads 16 are included in the ECG machine 15. The electrode pads 16 are positioned on a left chest area of the examinee S (patient body) at suitable points. The ECG machine 15 creates an electrocardiogram (ECG) of FIG. 14 of the examinee S according to a signal from the electrode pads 16. The ECG machine 15 transmits ECG information of the ECG to the diagnosis support apparatus 10.

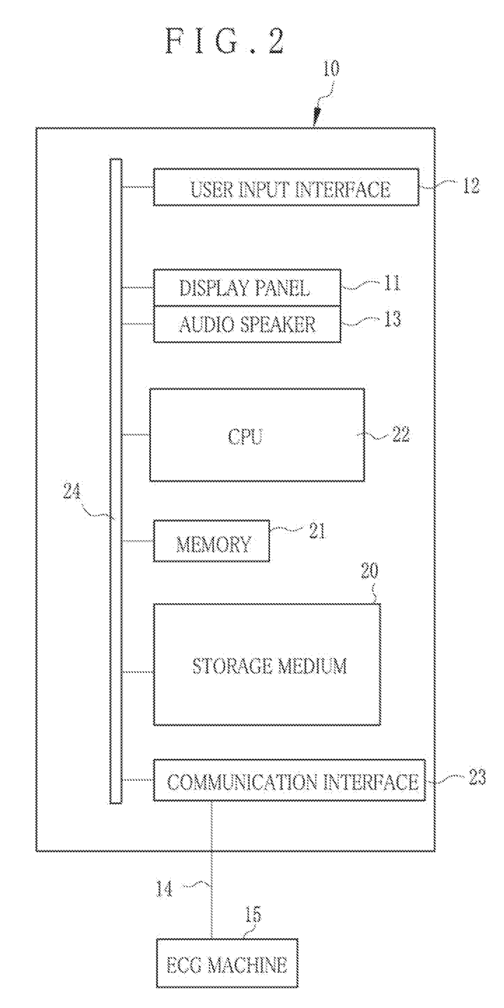

In FIG. 2, the diagnosis support apparatus 10 includes the display panel 11, the user input interface 12 and the audio speakers 13 and also a storage medium 20 or storage device, a memory 21, a CPU 22 or central processing unit, and a communication interface 23. A data bus 24 connects those circuit devices together.

The storage medium 20 is a hard disk drive incorporated in the diagnosis support apparatus 10, or connected to the diagnosis support apparatus 10 by a cable, network or the like. Also, the storage medium 20 may be a disk array having plural hard disk drives. The storage medium 20 stores a control program and various application programs such as the Operating System (OS), and relevant data associated with the programs.

The memory 21 is a working memory with which the CPU 22 performs tasks. The CPU 22 loads the memory 21 with the programs stored in the storage medium 20, and controls the various circuit devices in the diagnosis support apparatus 10 by performing the tasks according to the program.

The communication interface 23 is a USB interface for receiving ECG information of the ECG generated by the ECG machine 15 through the USB cable 14. The display panel 11 displays a control page according to the ECG information. The control page has a function according to the GUI (graphical user interface). The diagnosis support apparatus 10 receives manual inputs from the user input interface 12 by use of the various menus in the control page.

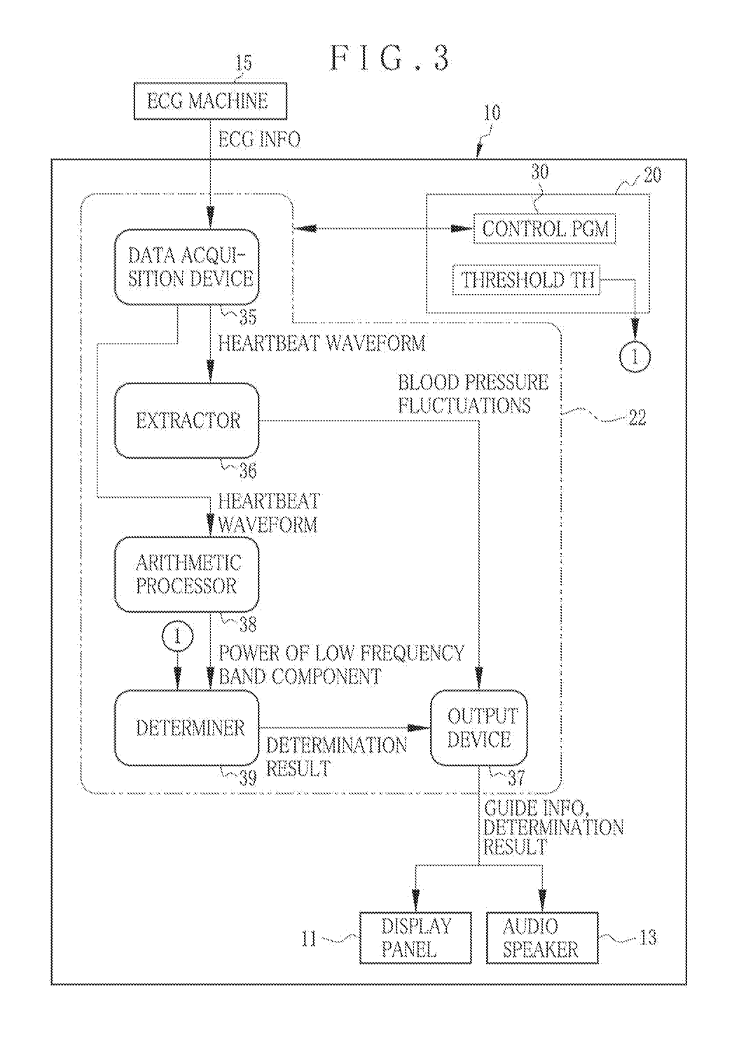

In FIG. 3, a control program 30 (computer-executable program) is stored in the storage medium 20. The control program 30 is an application program to cause a computer to function as a diagnosis support apparatus. Also, data of a threshold TH is stored in the storage medium 20 for use in determining occurrence or non-occurrence of a problem in autonomic activity of the autonomic nervous system in the examinee S.

Running the control program 30 causes the CPU 22 to function with a data acquisition device 35 or waveform generator, an extractor 36, an output device 37 or data generator or output controller, an arithmetic processor 38 and a determiner 39 in cooperation with the memory 21.

The data acquisition device 35 performs an acquiring function and acquires ECG information of the ECG of the examinee S from the ECG machine 15 to generate information of a heartbeat waveform. The data acquisition device 35 sends the heartbeat waveform to the extractor 36 and the arithmetic processor 38.

The extractor 36 performs extraction of blood pressure fluctuations based on changes in the blood pressure of the examinee S from a heartbeat waveform. The extractor 36 sends the blood pressure fluctuations to the output device 37.

The output device 37 is a guide information output device, and outputs guide information for inducing breathing of the examinee S for setting respiratory cycle time according to a period of blood pressure fluctuations. The output device 37 outputs the guide information to the display panel 11 and the audio speakers 13. In short, the output device 37 outputs the guide information in a visual form and audio form.

The arithmetic processor 38 performs an obtaining function of obtaining a power value of a low frequency band component of a heartbeat waveform (average energy per unit time of the low frequency band component) as a value related to the intensity of the heartbeat waveform. The arithmetic processor 38 outputs the power value of the low frequency band component to the determiner 39.

The determiner 39 performs a determining function to judge occurrence of a problem in autonomic activity of the autonomic nervous system in the examinee S. The determiner 39 performs comparison of a power value of the low frequency band component and a threshold TH. The determiner 39 sends a determination result to the output device 37.

The output device 37 also performs a result output function to output a determination result of the determiner 39 in addition to the guide information output function. In short, the output device 37 is also a result output device. The output device 37 sends the determination result to the display panel 11.

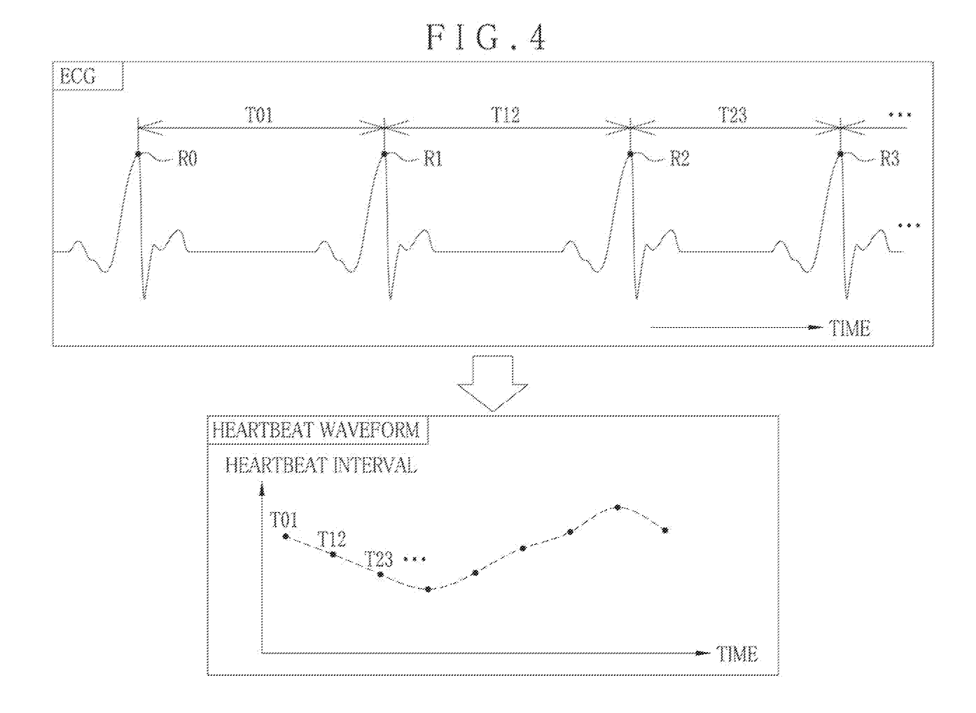

Details of the functions of the circuit devices 35-39 in the CPU 22 are described now by referring to FIGS. 4-13.

In FIG. 4, the data acquisition device 35 creates a heartbeat waveform according to ECG information of the ECG from the ECG machine 15. As is well-known medically, P, Q, R, S and T peaks appear in the ECG of one heartbeat. The data acquisition device 35 recognizes a heartbeat interval of the heartbeat from intervals of T01, T12, T23 and so on (referred to as RR intervals) between R0, R1, R2 and R3 peaks and so on among the peaks. The heartbeat waveform is created by plotting the intervals of T01, T12, T23 and so on with the axis of the timeline.

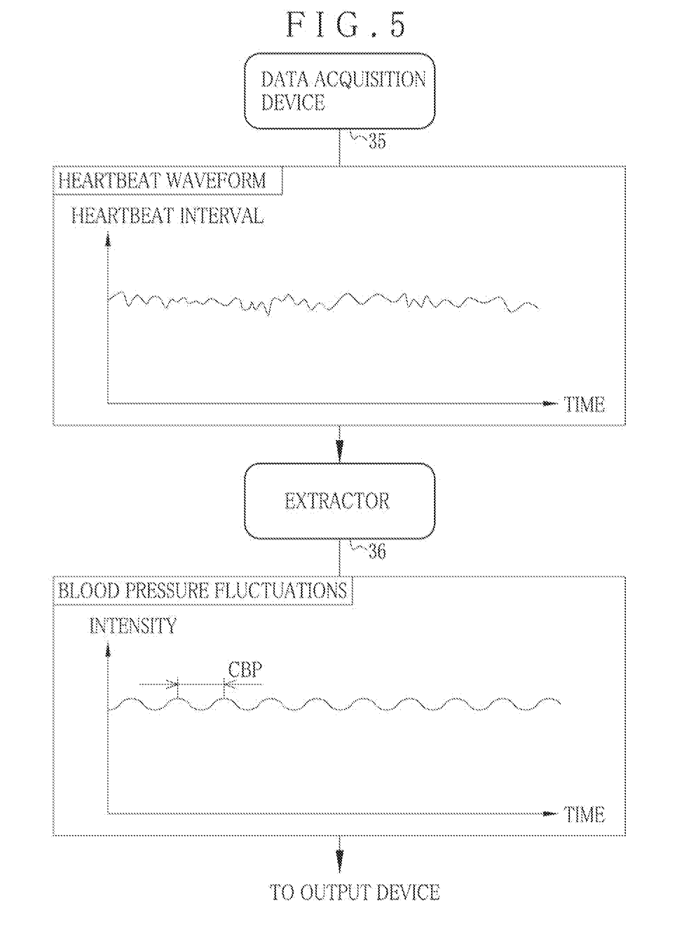

In FIG. 5, the extractor 36 processes the heartbeat waveform from the data acquisition device 35 in the well-known digital bandpass filter processing by use of the Fourier transform, inverse Fourier transform or the like. A range of the filter processing is set for a low frequency band component in a frequency range of 0.05-0.15 Hz, to extract the blood pressure fluctuations of the examinee S in the heartbeat waveform. Let CBP be the period of the blood pressure fluctuations.

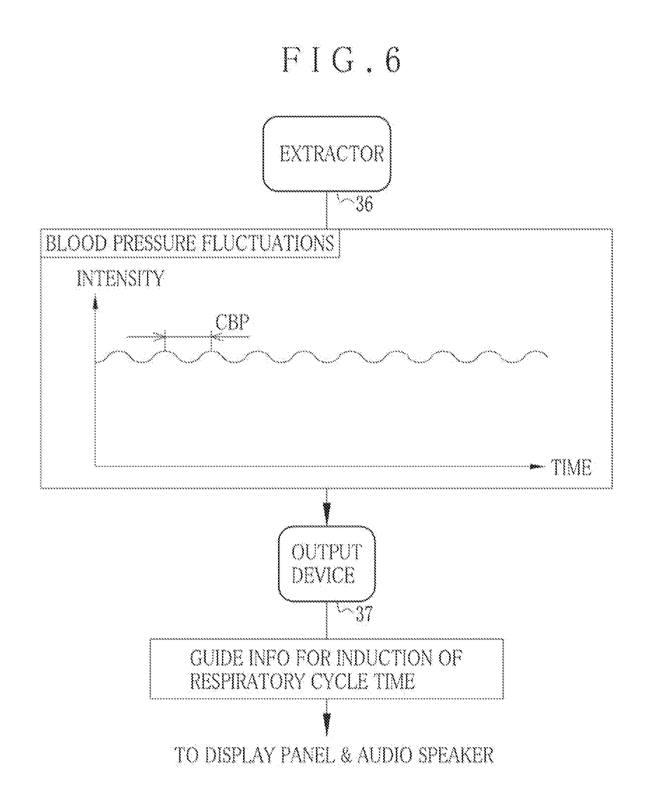

In FIG. 6, the output device 37 outputs guide information to the display panel 11 and the audio speakers 13 for induction of the examinee with the respiratory cycle time according to the period CBP of the blood pressure fluctuations from the extractor 36.

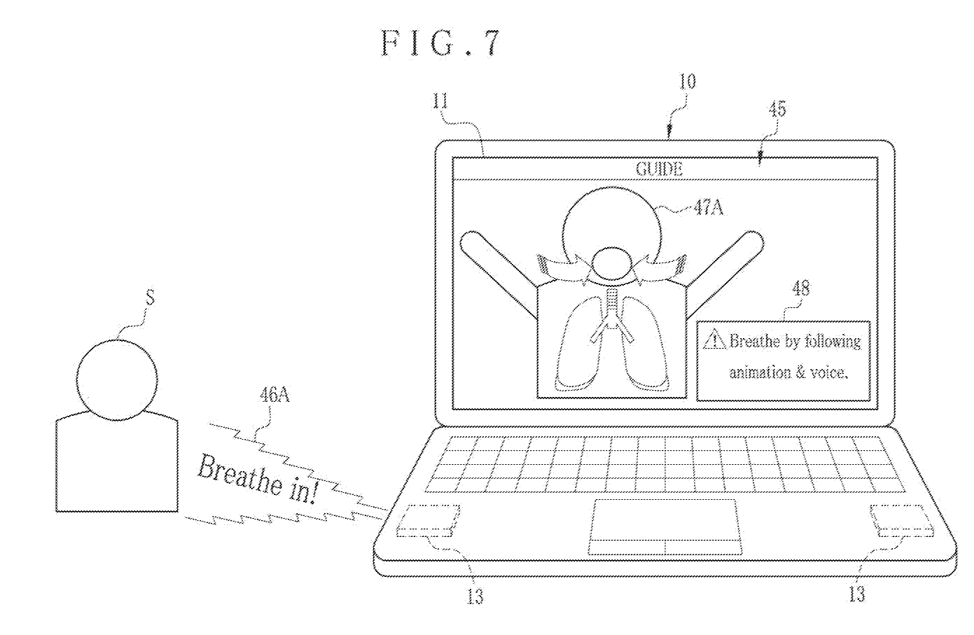

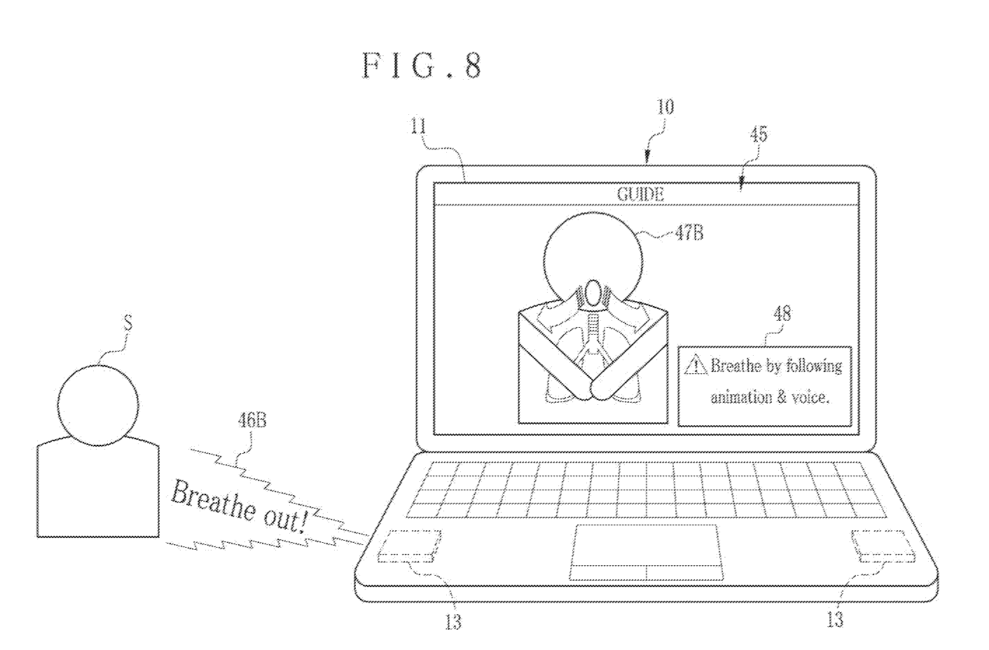

In FIGS. 7 and 8, a guide page 45 or guide screen is displayed on the display panel 11 after outputting of the output device 37. Guide voices 46A and 46B are emitted by the audio speakers 13. An example of the guide voice 46A is “Breathe in!” for instructing the examinee S to aspirate in FIG. 7. An example of the guide voice 46B is “Breathe out!” for instructing the examinee S to respire in FIG. 8.

Image animations 47A and 47B and an instruction message 48 are displayed in the guide page 45. In FIG. 7, the image animation 47A is an image of the examinee S who raises his or her arms, and lungs are expanded by oral aspiration. The image animation 47A is associated with the guide voice 46A to induce the examinee to aspirate. In FIG. 8, the image animation 47B is an image of the examinee S who lowers his or her arms, and lungs are contracted upon oral respiration. The image animation 47B is associated with the guide voice 46B to induce the examinee to respire. The instruction message 48 is a text to induce the examinees to breathe by following the image animations 47A and 47B and the guide voices 46A and 46B.

The output device 37 sets each one of the output time of the guide information of FIG. 7 and the output time of the guide information of FIG. 8 equal to a half of the period CBP of the blood pressure fluctuations. Thus, the output device 37 sets a sum of the output time of the guide information of FIG. 7 and the output time of the guide information of FIG. 8 equal to the period CBP of the blood pressure fluctuations. Note that inequality between the values of the output time can remain. For example, the output time of the guide information of FIG. 8 can be set longer than the output time of the guide information of FIG. 7.

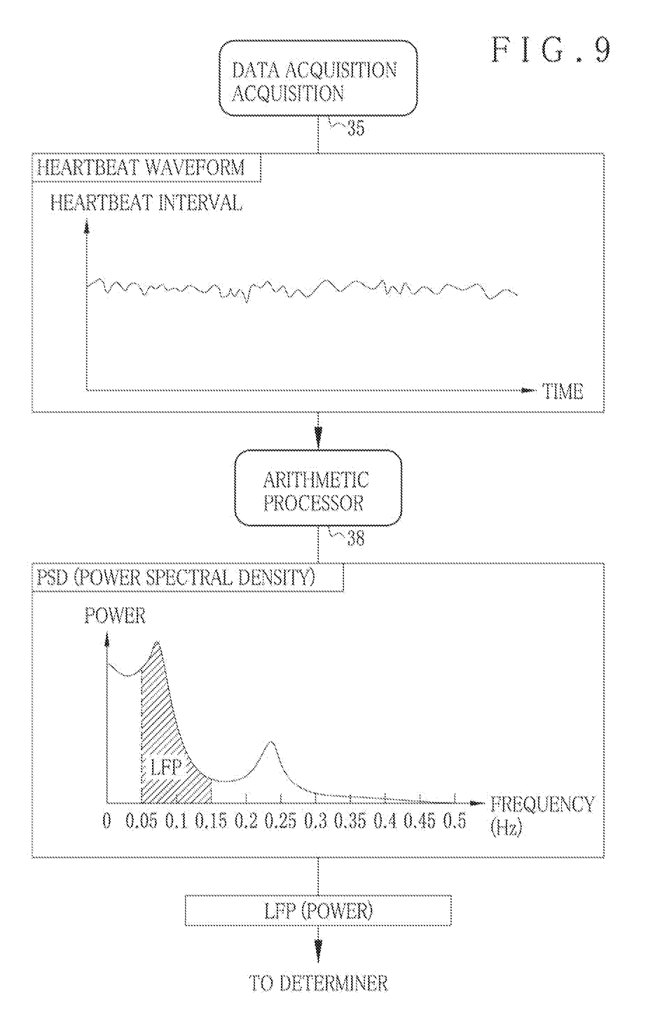

In FIG. 9, the arithmetic processor 38 obtains power spectral density (PSD) of the heartbeat waveform from the data acquisition device 35 by use of a well-known analysis algorithm, such as the autoregressive model. The arithmetic processor 38 obtains an integration value LFP of the power value in a range (of a frequency of 0.05-0.15 Hz) of the low frequency band component of the PSD as a power value of the low frequency band component.

Note that a frequency band of the power value LFP obtained in the arithmetic processor 38 is in a region of the frequency range of 0.05-0.15 Hz in the same manner as the blood pressure fluctuations of the examinee S extracted by the extractor 36. The respiratory cycle time is set equal to the period CBP of the blood pressure fluctuations according to the guide information to be described later. Thus, the low frequency band component of the heartbeat waveform comes to include not only the blood pressure fluctuations but also the respiratory fluctuations based on changes in the breathing of the examinee S. Note that a term of the “blood pressure fluctuations” is used to express a component extracted by the extractor 36. A term of the power value LFP of the “low frequency band component” is used to express the power value LFP obtained by the arithmetic processor 38.

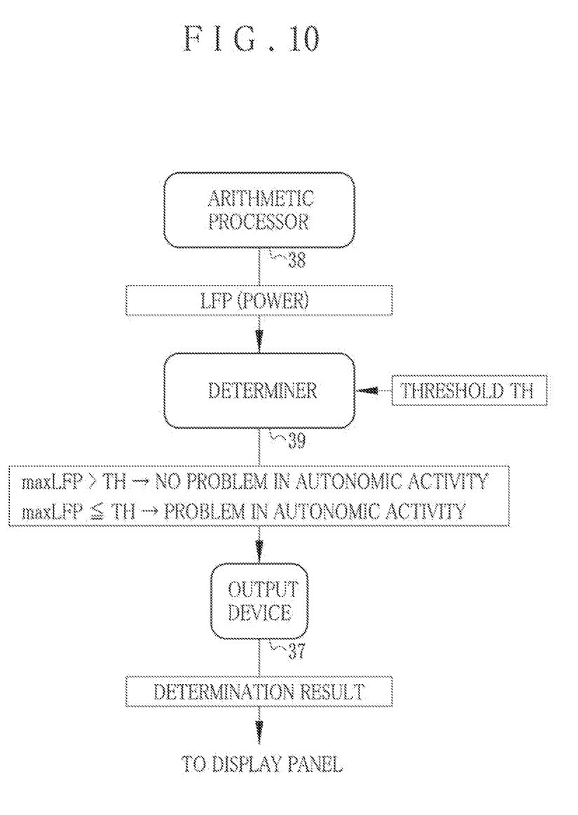

In FIG. 10, the determiner 39 compares a threshold TH and a maximum value maxLFP of the power value LFP of the low frequency band component from the arithmetic processor 38. Assuming that the maximum value maxLFP is higher than the threshold TH, the determiner 39 judges non-occurrence of a problem in autonomic activity in the autonomic nervous system in the examinee S. Assuming that the maximum value maxLFP is equal to or lower than the threshold TH, the determiner 39 judges occurrence of a problem in autonomic activity in the autonomic nervous system in the examinee S.

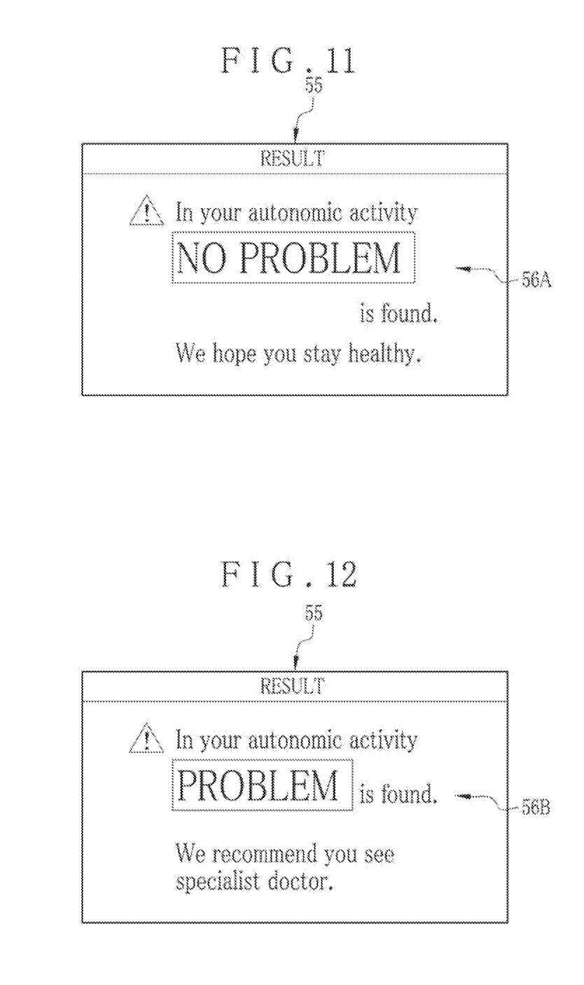

In FIGS. 11 and 12, the output device 37 causes the display panel 11 to display a result page 55 as an output form of the determination result of the determiner 39. In FIG. 11, a result message 56A is displayed to inform no occurrence of a problem in autonomic activity in the autonomic nervous system. In FIG. 12, a result message 56B is displayed to inform occurrence of a problem in autonomic activity in the autonomic nervous system, to recommend that the examinee should consult a specialist doctor and should be examined in a thorough examination.

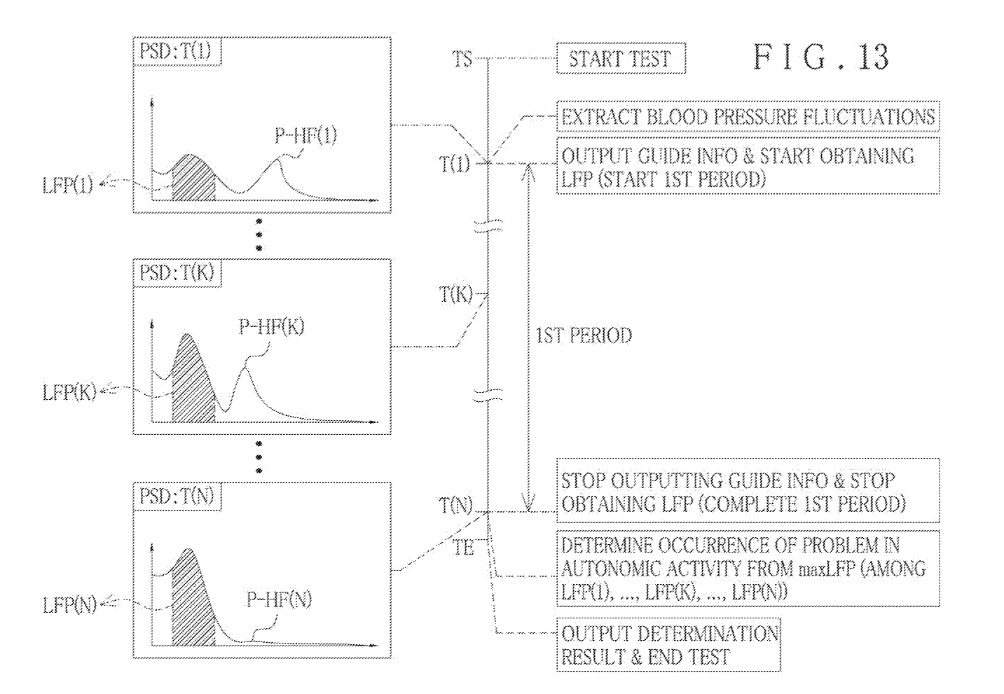

In FIG. 13, the maximum value maxLFP of the power value LFP of the low frequency band component is the maximum among the plural power values LFP (1), . . . , LFP (K), . . . , LFP (N) of the low frequency band component obtained by the arithmetic processor 38 during the first period from the time T (1) to the time T (N). Note that K=1, 2, 3, . . . , N−2, N−1, N, and N is a sample number of sampling of the power value LFP of the low frequency band component in the first period. T (K)-T (K−1) is a sampling period of the power value LFP of the low frequency band component.

At the time T (1) of starting the first period, the extractor 36 extracts the blood pressure fluctuations. The output device 37 starts outputting the guide information. The arithmetic processor 38 starts obtaining the power value LFP of the low frequency band component. Also, at the time T (N) of ending the first period, the output device 37 stops outputting the guide information. The arithmetic processor 38 stops obtaining the power value LFP of the low frequency band component. An example of the first period is in a range of 3-10 minutes sufficient for relaxing the body of the examinee S.

At the time T (1), outputting the guide information is started. The respiratory cycle time is not equal to the period CBP of the blood pressure fluctuations. There is no relaxation of the examinee S. The power value of the region of the low frequency band component is relatively small as indicated with PSD:T (1) at the time T (1), so that the LFP (1) is relatively small. Also, a peak P-HF (1) occurs because of the respiratory fluctuations based on the fluctuations of breathing of the examinee S in a high frequency band of 0.15-0.4 Hz.

At the time T (K) later than the time T (1) by a predetermined duration, the respiratory cycle time becomes near to the period CBP of the blood pressure fluctuations as a result of induction with the guide information. The power value of the region of the low frequency band component becomes large in the relaxation in comparison with that at the time T (1), as indicated with PSD:T (K) at the time T (K), so that the value LFP (K) becomes large. Also, a peak P-HF (K) occurs because of the respiratory fluctuations, with a higher power value than that of the peak P-HF (1) at the time T (1) and nearer to a band of the low frequency band component.

At the time T (N), the respiratory cycle time is equal to the period CBP of the blood pressure fluctuations, so that the examinee S is relaxed. A peak P-HF (N) owing to the respiratory fluctuations disappears substantially and becomes absorbed in the low frequency band component, which is in a condition of PSD:T (N) at the time T (N). Thus, the power in a region of the low frequency band component is increased, and LFP (N) is also increased.

The determiner 39 does not perform determination before the lapse of the first period. After the first period, the determiner 39 selects the maximum value maxLFP among the plural power values LFP (1), . . . , LFP (K), . . . , LFP (N) of the low frequency band component input by the arithmetic processor 38 during the first period.

Specifically, the determiner 39 has a memory for storing the power value LFP of the low frequency band component, and performs comparison between the power value LFP (K−1) of the low frequency band component input by previous sampling and the power value LET (K) of the low frequency band component input by current sampling at each time that the power value LET of the low frequency band component is input by the arithmetic processor 38. Assuming that LFP (K−1)<LFP (K), then the determiner 39 overwrites the power value LFP (K) over the power value LFP (K−1) stored in the memory by rewriting. Assuming that LFP (K−1)≧LFP (K), then no rewriting is performed. Thus, the power value LET of the low frequency band component stored in the memory after the lapse of the first period becomes the maximum value maxLFP.

The time TS is time of manipulating the user input interface 12 by the operator of the diagnosis support apparatus 10 to instruct the ECG machine 15 to start the test. The time TE is time of outputting the determination result of the determiner 39 at the output device 37, and completing the test. A time interval occurs between the time TS and time T (1), in order that the sample number of sampling of the RR interval required for creating the heartbeat waveform will be kept sufficiently, and that the precision will be high in extracting the heartbeat waveform as resource of the guide information. An example of the time interval between the time TS and time T (1) is one minute. Also, a time interval occurs between the time T (N) and time TE in requirement for the determination in the determiner 39 and outputting the determination result in the output device 37. An example of the time interval is in a range from several tens of microseconds to several milliseconds.

The operation of the above construction is described now by referring to a flow in FIG. 14. At first, an operator sets the electrode pads 16 of the ECG machine 15 to suitable points on the left chest area of the examinee S, and manipulates the user input interface 12 to start the ECG machine 15 for a diagnostic test at the time TS. The ECG machine 15 starts operation, and sends information of an ECG of the examinee S to the diagnosis support apparatus 10. The data acquisition device 35 acquires the ECG information of the ECG at a step S100, and generates a heartbeat waveform according to the ECG.

Upon the lapse of the predetermined time from the instruction of starting the test (at the time T (1)), the extractor 36 extracts a blood pressure fluctuations of the examinee S from the heartbeat waveform at a step S110 or extracting step. The output device 37 starts outputting the guide information for inducing breathing of the respiratory cycle time equal to the period CBP of the blood pressure fluctuations, at a step S120 or guide information output step. At the same time, the arithmetic processor 38 starts obtaining the power value LFP of the low frequency band component at the step S120 or obtaining step.

As the guide information for inducing the respiratory cycle time equal to the period CBP of the blood pressure fluctuations of the examinee S, it is possible to set the respiratory cycle time by considering a specificity in the period of the blood pressure fluctuations. Any one of the examinees S can become in relaxation, as the respiratory cycle time according to the guide information can become suitable for each of the examinees S.

The guide information is output by the display panel 11 and the audio speakers 13 in the forms of the guide page 45 and the guide voices 46A and 46B. The examinee S can receive the guide information both visually and acoustically, and can be induced to set his or her respiratory cycle time according to the period CBP of the blood pressure fluctuations smoothly.

Outputting the guide information from the output device 37 is repeated in the first period in (no in a step S130). Thus, the examinee S is caused to become relaxed gradually. Also, obtaining the power value LFP of the low frequency band components in the arithmetic processor 38 is repeated in the first period at a predetermined sampling period (no in the step S130). Thus, the determiner 39 is successively supplied with the plural power values LFP (1), . . . , LFP (K), . . . , LFP (N) of the low frequency band components obtained by the arithmetic processor 38 in the first period.

Upon the lapse of the first period at the time T (N) (yes in the step S130), the output device 37 stops outputting the guide information. The arithmetic processor 38 stops obtaining the power value LFP of the low frequency band component in a step S140.

Upon the lapse of the first period, the determiner 39 selects a maximum value maxLFP among the plural power values LFP (1), . . . , LFP (K), . . . , LFP (N) of the low frequency band components. The selected maximum value maxLFP is compared with the threshold TH, to determine occurrence of a problem in autonomic activity (physiological changes) in the autonomic nervous system in the examinee S in a step S150 or determining step.

As has been described with FIG. 13, the power value LFP becomes comparatively high in case the examinee S becomes relaxed after obtaining equality between the period CBP of the blood pressure fluctuations and the respiratory cycle time. The maximum value maxLFP can be regarded as the power value LET of the low frequency band component while the examinee S is in the most relaxed state in the first period. It is possible to prevent changes in the heartbeat waveform due to stress because the determination is performed for occurrence of a problem in autonomic activity in the examinee S according to the maximum value maxLFP. Occurrence of a problem in autonomic activity in the examinee S can be recognized with considerably high precision in the determination.

In case the respiratory cycle time becomes equal to the period CBP of the blood pressure fluctuations to relax the examinee S, the respiratory fluctuations are decreased nearly to zero and absorbed in the low frequency band component. The power value LFP of the low frequency band component becomes a relatively high value. Also, the maximum value maxLFP becomes higher. Thus, it is possible to perform the determination precisely and easily, because of the determination according to the power value LET of the low frequency band component after obtaining the power value LFP relevant to the intensity of the heartbeat waveform (intensity level of signal intensity), in comparison with determination based on a relatively small value even while the examinee S is relaxed. Furthermore, the determination can be clarified and requires only short time, because of the comparison between the maximum value maxLFP and the threshold TH.

Also, any one of the examinees S becomes in relaxation before performing the determination. It is possible to perform the determination in an equal condition between the examinees S, and to evaluate the determination results of the examinees in comparison in equal criteria.

The determination result is output by the output device 37 to the display panel 11 in the form of the result page 55 in a step S160 or output step. Thus, the diagnostic test is completed at the time TE.

The examinee S can be informed of occurrence or non-occurrence of a problem in autonomic activity by the result page 55. A doctor or operator can perform medical care or treatment appropriately to the examinee S by assistance of the result page 55. Particularly assuming that the result page 55 of FIG. 12 for the occurrence of the problem in autonomic activity is displayed, the examinee can immediately talk with the doctor to request medical care of a specialist doctor or request a diagnostic test in a thorough examination.

### Second Embodiment

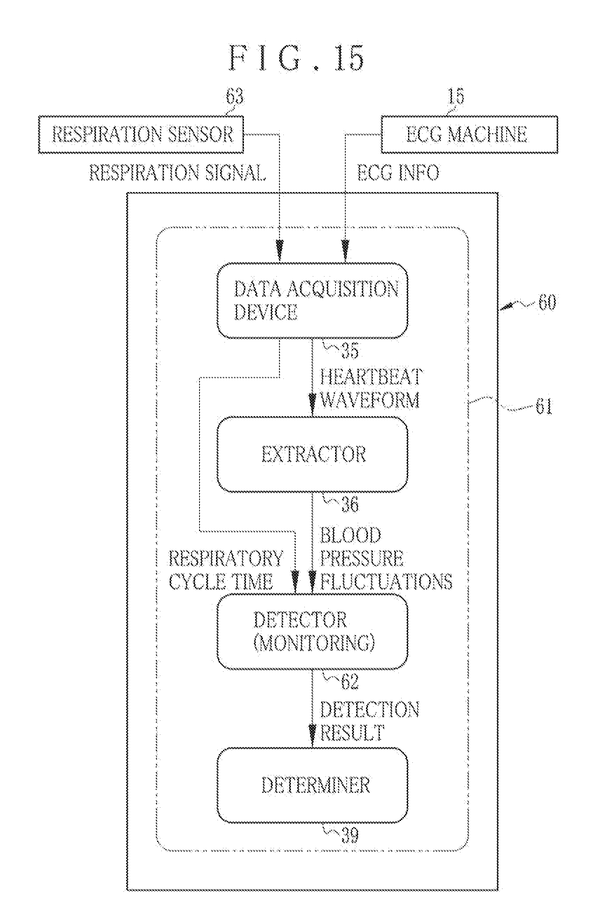

In contrast with the first embodiment of utilizing the maximum value maxLFP of the power value LFP, breathing of the examinee S (patient body) is detected in a second embodiment of FIGS. 15-17, to check whether the period CBP of the blood pressure fluctuations is approximately equal to the respiratory cycle time. The determination is performed assuming that a condition of the approximate equality is continued in a second period as a predetermined period.

In a diagnosis support apparatus 60 for physiological monitoring in FIG. 15, a detector 62 or monitoring device is established in a CPU 61 or central processing unit in addition to the circuit devices 35-39 of the first embodiment (among which the output device 37 and the arithmetic processor 38 are not shown). The detector 62 checks whether the period CBP of the blood pressure fluctuations of the examinee S is approximately equal to the respiratory cycle time, and sends a detection result of checking to the determiner 39.

The data acquisition device 35 acquires a respiration signal from a respiration sensor 63 in addition to the ECG information of the ECG of the examinee S from the ECG machine 15. The data acquisition device 35 obtains respiratory cycle time according to the respiration signal, and outputs the information of the period to the detector 62. Also, the extractor 36 outputs information of the blood pressure fluctuations to the detector 62 as well as the output device 37.

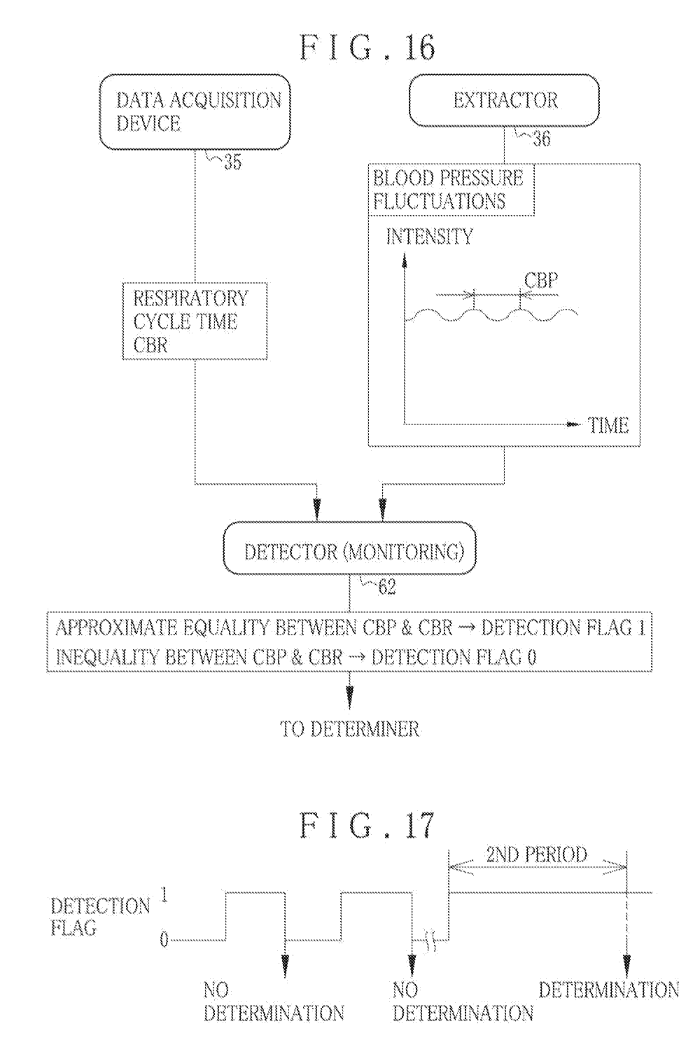

In FIG. 16, the detector 62 checks whether the period CBP of the blood pressure fluctuations from the extractor 36 is approximately equal to the respiratory cycle time CBR from the data acquisition device 35. Assuming that the respiratory cycle time CBR is in a range of the period CBP±α(CBP−α≦CBR≦CBP+α), then the detector 62 detects that the period CBP of the blood pressure fluctuations is approximately equal to the respiratory cycle time CBR, and sends a detection flag of “1” to the determiner 39. Assuming that the respiratory cycle time CBR is not in the range of the period CBP±α(CBR<CBP−α or CBR>CBP+α), then the detector 62 detects that the period CBP of the blood pressure fluctuations is unequal to the respiratory cycle time CBR, and sends a detection flag of “0” to the determiner 39.

Note that an example of α is in a range from several hundreds of milliseconds to several seconds. The value of α is used as a margin for regarding equality of the respiratory cycle time CBR with the period CBP of the blood pressure fluctuations even though the respiratory cycle time CBR is not strictly equal with the period CBP of the blood pressure fluctuations. Note that α may be equal to zero (0). In other words, the value of the margin may not be predetermined. For this configuration, a detection flag of “1” is output only in case CBP=CBR.

In FIG. 17, the determiner 39 does not perform the determination before the detection result of approximate equality between the period CBP of the blood pressure fluctuations and the respiratory cycle time CBR, namely the detection flag of 1, is generated by the detector 62 in a continuous manner in the second period predetermined for reference. In case the detection flag of 1 is generated by the detector 62 in a continuous manner in the second period, the determiner 39 performs the determination according to the power value LFP of the low frequency band component received from the arithmetic processor 38. In the same manner as the first period, an example of the second period is in a range of 3-10 minutes sufficient for relaxing the examinee S.

In the first embodiment, no detection is performed for approximate equality between the period CBP of the blood pressure fluctuations of the examinee S and the respiratory cycle time CBR. Incorrect determination is likely to occur with remaining inequality between the period CBP of the blood pressure fluctuations of the examinee S and the respiratory cycle time CBR as the guide information may not be considered by the examinee S. However, it is possible in the present embodiment to obtain high precision in the determination, as the determination is performed while the period CBP of the blood pressure fluctuations of the examinee S is reliably set approximately equal to the respiratory cycle time CBR.

Also, the determination is performed after the approximate equality between the period CBP of the blood pressure fluctuations and the respiratory cycle time CBR is continued for the second period. The determination can be performed while the examinee S is relaxed with certainty. Noise created in determining the problem in autonomic activity due to stress can be eliminated reliably.

Examples of the respiration sensor 63 include a sensor positioned in a nostril of the examinee S for detecting breathing from a temperature difference between aspiration and respiration, or a change in air pressure between those, and a sensor positioned on the chest of the examinee S for detecting breathing from movement of the chest. Also, the breathing can be detected according to the ECG information of the ECG from the ECG machine 15 on the basis of utilization of changes of the amplitude of the ECG depending upon the breathing.

### Third Embodiment

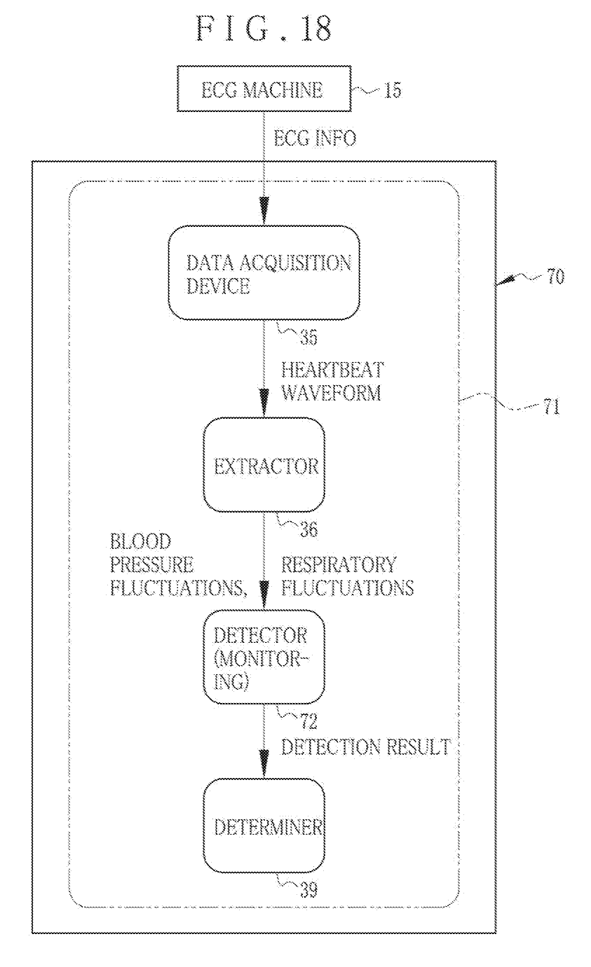

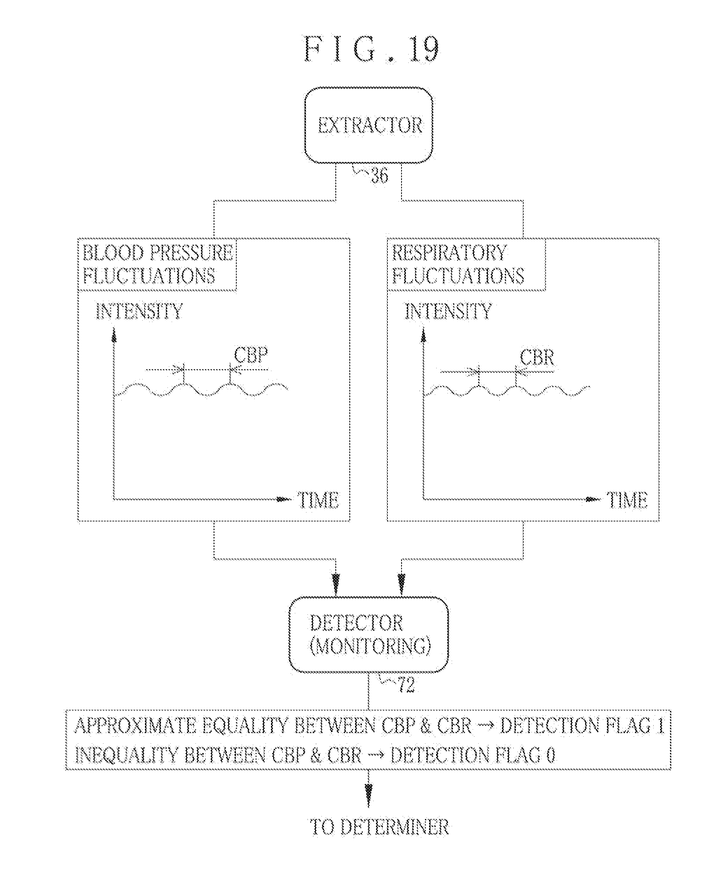

In contrast with the second embodiment of performing the detection according to the respiration signal from the respiration sensor 63, another preferred embodiment is illustrated in FIGS. 18 and 19 in which respiratory fluctuations are also extracted from the heartbeat waveform in addition to the blood pressure fluctuations, and are utilized for the detection.

In FIG. 18, a CPU 71 or central processing unit in a diagnosis support apparatus 70 for physiological monitoring includes a detector 72 or monitoring device established therein in the same manner as the second embodiment. The respiration sensor 63 of the second embodiment is not used in the present embodiment. Instead of this, the extractor 36 processes the heartbeat waveform in digital bandpass filter processing well-known in the art, and extracts information of respiratory fluctuations in addition to the blood pressure fluctuations from the heartbeat waveform (high frequency band component in a frequency range of 0.15-0.4 Hz). The extractor 36 outputs the information of the blood pressure fluctuations and the respiratory fluctuations to the detector 72.

In FIG. 19, the detector 72 checks whether the period CBP of the blood pressure fluctuations from the extractor 36 is approximately equal to the period CBR of the respiratory fluctuations from the extractor 36 (or the respiratory cycle time in the second embodiment). For further steps of the operation, the steps of the second embodiment are repeated.

In the embodiment, it is possible to save the manufacturing cost in relation to the absence of the respiration sensor 63, and to prevent the examinee S from having uncomfortable feeling with the respiration sensor 63, as effects additional to those of the second embodiment.

As has been described with FIG. 13, the peak P-HF owing to the respiratory fluctuations is decreased to decrease the power value of the respiratory fluctuations while the period CBP of the blood pressure fluctuations is equal to the respiratory cycle time CBR of the respiratory fluctuations. It is possible to utilize this feature of the decrease for checking equality between the period CBP of the blood pressure fluctuations and the respiratory cycle time CBR of the respiratory fluctuations, instead of the direct comparison between the period CBP of the blood pressure fluctuations from the extractor 36 and the respiratory cycle time CBR of the respiratory fluctuations from the extractor 36.

Specifically, the power value of the high frequency band component in the frequency range of 0.15-0.4 Hz is obtained by the arithmetic processor 38 in the same manner as the power value LFP of the low frequency band component. The obtained power value of the high frequency band component is compared with the predetermined threshold. Assuming that the power value of the high frequency band component is lower than the threshold, then it is detected that the period CBP of the blood pressure fluctuations is equal to the respiratory cycle time CBR of the respiratory fluctuations.

In the second and third embodiments, assuming that it is detected that the period CBP of the blood pressure fluctuations is equal to the respiratory cycle time CBR and assuming that the detection flag of 1 is output to the determiner 39, it is likely that the period CBP of the blood pressure fluctuations is unequal instantaneously to the respiratory cycle time CBR. Assuming that the period CBP of the blood pressure fluctuations immediately becomes equal again to the respiratory cycle time CBR, it is possible to continue outputting the detection flag of 1 to the determiner 39. This is effective in preventing output of a detection flag of zero (0) with instantaneous inequality between the period CBP of the blood pressure fluctuations and the respiratory cycle time CBR in advance of continuation of the detection flag of 1 for the second period. Delay of the determination can be prevented.

Assuming that it is detected in the second and third embodiments that the period CBP of the blood pressure fluctuations is greatly unequal to the respiratory cycle time CBR or period CBR of the respiratory fluctuations in the detector 62 or 72, then it is possible to output voice or sound through the audio speakers 13 for inducing the examinee to breathe with his or her respiratory cycle time according to the guide information.

Also, the operator or the examinee S can monitor approximate equality of the period CBP of the blood pressure fluctuations and the respiratory cycle time CBR by himself or herself. He or she can manipulate the user input interface 12 for inputting information of the time of the determination.

### Fourth Embodiment

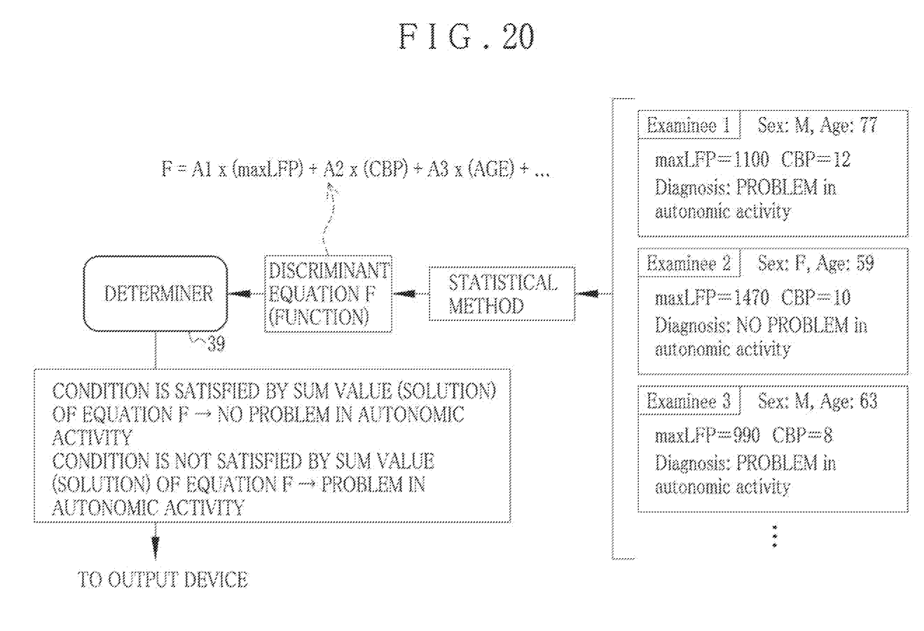

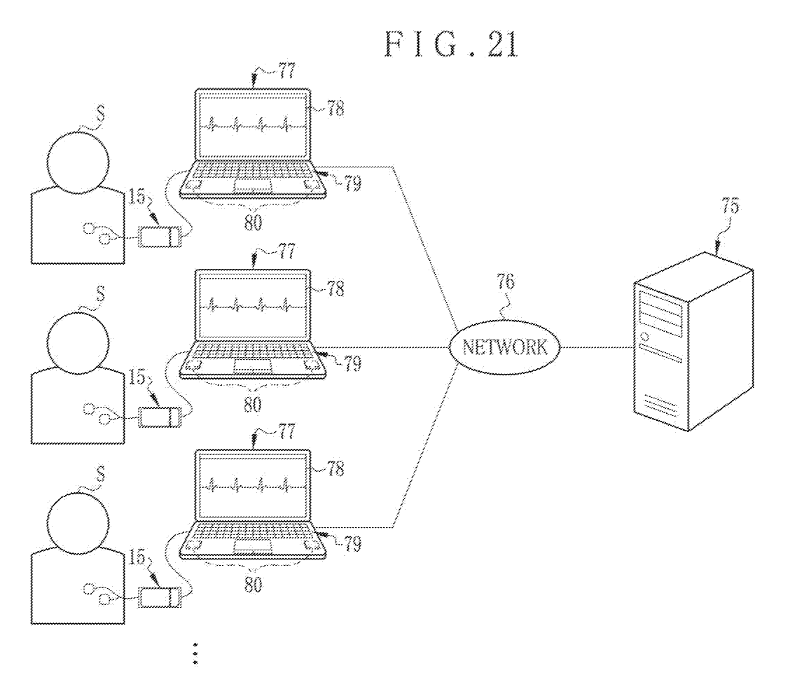

In the first embodiment, the determination is performed according to the comparison between the threshold TH and the maximum value maxLFP of the power value LFP of the low frequency band component. In contrast, the determination is performed according to a discriminant equation in FIG. 20.

In FIG. 20, the determiner 39 of the embodiment performs the determination according to a discriminant equation F. The storage medium 20 stores the discriminant equation F instead of the threshold TH. The discriminant equation F is an expression obtained by a statistical method of linear regression analysis or logistic regression analysis according to past cases accumulated medically, including Similar Cases 1, 2, 3 and so on. The Similar Cases 1, 2, 3 and so on are stored in EMRs managed in the medical facility. Examples of data included in the information in Similar Cases 1, 2, 3 and so on include sex, age and other personal data of the examinee S, the maximum value maxLFP of the power value LFP of the low frequency band component obtained by the diagnosis support apparatus 10 and the period CBP of the blood pressure fluctuations, a doctor's diagnosis result of occurrence or non-occurrence of a problem in autonomic activity in the autonomic nervous system, and the like.

An example of the discriminant equation F is Equation (1):

F=A1×(maxLFP)+A2×(CBP)+A3×(age)+ . . . Equation (1):

In short, the discriminant equation F (multi variable function) is a function with at least a variable of the maximum value maxLFP of the power value LFP of the low frequency band component. Variables other than the maximum value maxLFP are the period CBP of the blood pressure fluctuations, the age of the examinee S and the like. A1, A2, A3 and so on are coefficients.

The determiner 39 substitutes particular values for the variables in the discriminant equation F (multi variable function), and obtains a sum value or parameter value (solution) according to the discriminant equation F. Assuming that the sum value from the discriminant equation F satisfies a predetermined condition, then the determiner 39 judges occurrence of a problem in autonomic activity. Assuming that the sum value from the discriminant equation F does not satisfy the predetermined condition, then the determiner 39 judges non-occurrence of a problem in autonomic activity. An example of the predetermined condition is a value range defined by upper and lower threshold values, with which it is checked whether the sum value is within the value or not. For further steps of the operation, the steps of the first embodiment are repeated. Consequently, it is possible to obtain high reliability in the determination, because the discriminant equation F is used for the determination on the basis of the statistical method in combination of the past cases accumulated medically in a large scale.

In the above embodiments, the ECG information of the ECG of the examinee S from the ECG machine 15 is acquired as information of the heartbeat waveform. Alternatively, a signal of a heart rate from a heart rate sensor or a signal of a pulse rate from a pulse sensor can be acquired as information of the heartbeat waveform. Also, the ECG machine 15 can be provided with a function for creating a heartbeat waveform in contrast with the embodiments of creating the heartbeat waveform in the data acquisition device 35 according to the ECG information.

The output form of the guide information is not limited to the first embodiment with the guide page 45 at the display panel 11 and the guide voices 46A and 46B at the audio speakers 13. It is possible to use only one of the guide page 45 displayed on the display panel 11 and the guide voices 46A and 46B emitted by the audio speakers 13. Also, messages for inducing the examinee S to aspirate and respire (breathe in and out) can be displayed in place of the image animations 47A and 47B. Furthermore, a vibrator can be used for manual touch of the examinee S, to inform the guide information by vibrational indication, for example, the vibrator can be driven to vibrate upon aspiration of the examinee S.

In the above embodiment, the output device 37 performs the guide information output function and the result output function. However, two discrete output devices can be used, a first one of those being used for the guide information output function, a second one of those being used for the result output function. Also, the determination result can be output with voice or sound in addition to the guide information. Furthermore, the determination result can be printed as an output.

Also, a method of obtaining the power value LFP of the low frequency band component is not limited to obtaining according to the PSD in the first embodiment. For example, the power value LFP can be obtained according to the blood pressure fluctuations extracted by the extractor 36.

Also, it is possible to obtain a power value of the heartbeat waveform including the power value LFP of the low frequency band component and the power value of the high frequency band component in relation to the intensity of the heartbeat waveform (intensity level of signal intensity) in place of the power value LFP of the low frequency band component. It is possible to obtain a ratio between the power value LFP of the low frequency band component and the power value of the high frequency band component as a value related to the intensity of the heartbeat waveform.

In the embodiments, the diagnosis support apparatus 10 is constituted by a notebook type of personal computer. However, the invention is not limited thereto. For example, the diagnosis support apparatus 10 can be constituted by a server computer as illustrated in FIG. 21.

In FIG. 21, a diagnosis support apparatus 75 for physiological monitoring is a server computer, and plural client terminal apparatuses 77 are connected to the diagnosis support apparatus 75 in a communicable manner by use of a network 76, for example, LAN (local area network) installed in a medical facility. The diagnosis support apparatus 75 has performance of simultaneously processing requests from the client terminal apparatuses 77.

The client terminal apparatuses 77 are disposed in respectively plural hospital rooms (consulting rooms) in the hospital (medical facility). Each one of the client terminal apparatuses 77 is combined with the ECG machine 15 as one set. The client terminal apparatuses 77 transmit the ECG information of the ECG from the ECG machine 15 to the diagnosis support apparatus 75 by use of the network 76.

Also, each of the client terminal apparatuses 77 includes a display panel 78, a user input interface 79 or input panel, and audio speakers 80 in the same manner as the diagnosis support apparatus 10. The display panel 78 displays the guide page 45 and the result page 55. The audio speakers 80 output the guide voices 46A and 46B. However, the client terminal apparatus 77 does not have a function of the diagnosis support, but has a function of transmitting the ECG information from the ECG machine 15 to the diagnosis support apparatus 75 and a function of displaying the guide information and determination result. The data acquisition device 35, the extractor 36, the output device 37, the arithmetic processor 38 and the determiner 39 of the above embodiments are established in the CPU of the diagnosis support apparatus 75.

To this end, the output device 37 in the diagnosis support apparatus 75 creates the guide page 45 and the result page 55 viewable on a web browser, and sends the guide page 45 and the result page 55 to the client terminal apparatus 77. The output device 37 of the diagnosis support apparatus 75 sends information of the guide voices 46A and 46B to the client terminal apparatus 77 in a format of audio outputs on the web browser.

The diagnosis support apparatus 75 sends an authorization key to the client terminal apparatus 77 and gives an access right to the diagnosis support apparatus 75. After the client terminal apparatus 77 performs access to the diagnosis support apparatus and receives the authorization, the diagnosis support apparatus 75 sends the guide page 45 and the result page 55 to the client terminal apparatus 77, which drives the display panel 78 to display the guide page 45 and the result page 55. The diagnosis support apparatus 75 sends signals of the guide voices 46A and 46B, which are output by the audio speakers 80.

The diagnosis support apparatus 75 outputs the guide page 45 and the result page 55 in a format of XML data for web delivery created according to the XML (Extensible Markup Language) as a markup language. The client terminal apparatus 77 performs display processing (display control) to display the guide page 45 and the result page 55 on the web browser according to the XML data (screen data). Also, it is possible to use another data description language instead of the XML, such as JSON (JavaScript Object Notation) and the like, JavaScript being a trade name.

Also, the data as resource of the guide page 45 and the result page 55 can be transmitted by the output device 37 of the diagnosis support apparatus 75 to the client terminal apparatus 77, which can create the guide page 45 or the result page 55.

Hardware construction of the computer for constituting the diagnosis support apparatus 75 of the present invention can be modified suitably. For example, the diagnosis support apparatus 75 can be constituted by a plurality of server computers discrete from one another for the purpose of increasing performance of processing and reliability. Specifically, a first server computer may constitute the data acquisition device 35, the extractor 36 and the arithmetic processor 38. A second server computer may constitute the output device 37 and the determiner 39. The diagnosis support apparatus 75 can be constituted by the two server computers.

In the above embodiments, the diagnosis support apparatus 75 is used in one medical facility. However, it is possible to use the diagnosis support apparatus 75 commonly in a plurality of medical facilities.

In the above embodiments, the client terminal apparatus 77 in one medical facility is connected to the diagnosis support apparatus 75 communicably by use of the network 76 such as the LAN. The diagnosis support apparatus 75 delivers the guide information and the determination result to the client terminal apparatus 77. To use the guide information and the determination result in the plural medical facilities, the diagnosis support apparatus 75 is set on-line with the plural client terminal apparatuses 77 positioned in the medical facilities by use of the wide area network (WAN), such as the Internet, public communication network and the like. Requests from the client terminal apparatuses 77 of the medical facilities are received by the diagnosis support apparatus 75 with the WAN, to deliver the guide information and the determination result to the client terminal apparatus 77. Note that information security should be established for use of the WAN, for example, the Virtual Private Network (VPN) or Hypertext Transfer Protocol Secure (HTTPS) can be preferably used as communication protocol of a high level of security.

A place of installation and manager of the diagnosis support apparatus 75 can be a data center of a service provider (company) separate from the medical facilities, but can be a suitable one of the plural medical facilities.

Note that the examinee is a patient of a disease (disorder, disease or injury). The diagnostic test is performed in the medical facility or hospital. However, an examinee in relation to the present invention can be a healthy body of a person. A diagnostic test may be health check-up.

The feature of the invention is used in the diagnostic test of the autonomic nerve function, but can be used in a diagnostic test of other functions of a living body, such as a diagnostic test of functions of a cardiovascular system, monitoring a rise or drop of physiological functions of a living body, and the like.

The present invention is not limited to the above embodiments. Various features of the embodiments and variants of the invention can be combined with each other suitably. Also, the computer-executable program and a non-transitory computer readable medium for storing the computer-executable program are included in the scope of the present invention.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A diagnosis support apparatus comprising:
a data acquisition device for obtaining information of a heartbeat waveform of an examinee;
an extractor for extracting blood pressure fluctuations based on changes of blood pressure of said examinee from said heartbeat waveform;
a guide information output device for outputting guide information for induction of respiratory cycle time of breathing of said examinee according to a period of said blood pressure fluctuations;
an arithmetic processor for obtaining a parameter value related to intensity of said heartbeat waveform;
a determiner for determining occurrence or non-occurrence of a problem in autonomic activity in an autonomic nervous system in said examinee according to said parameter value; and
a result output device for outputting a determination result of said determiner.

2. A diagnosis support apparatus as defined in claim 1, wherein said determiner performs said determining according to a maximum of said parameter value within a predetermined first period.

3. A diagnosis support apparatus as defined in claim 1, further comprising a detector for detecting whether said period of said blood pressure fluctuations corresponds to said respiratory cycle time of said examinee.

4. A diagnosis support apparatus as defined in claim 3, wherein said detector performs detection according to a signal from a respiration sensor for detecting breathing of said examinee.

5. A diagnosis support apparatus as defined in claim 3, wherein said extractor further extracts respiratory fluctuations based on changes in breathing of said examinee from said heartbeat waveform;
said detector performs detection according to said respiratory fluctuations.

6. A diagnosis support apparatus as defined in claim 3, wherein said determiner performs said determining in case a detection result of correspondence between said period of said blood pressure fluctuations and said respiratory cycle time of said examinee is continuously output by said detector for a predetermined second period.

7. A diagnosis support apparatus as defined in claim 1, wherein said guide information output device outputs said guide information in a visual form and/or audio form.

8. A diagnosis support apparatus as defined in claim 1, wherein said determiner performs said determining by comparison with said parameter value with a predetermined threshold.

9. A diagnosis support apparatus as defined in claim 1, wherein said determiner performs said determining according to a discriminant equation in which a variant is said parameter value and which is obtained statistically according to past cases.

10. A diagnosis support apparatus as defined in claim 1, wherein said arithmetic processor obtains a power value of a low frequency band component of said heartbeat waveform for said parameter value.

11. An operating method for a diagnosis support apparatus, comprising steps of:
obtaining information of a heartbeat waveform of an examinee;
extracting blood pressure fluctuations based on changes of blood pressure of said examinee from said heartbeat waveform;
outputting guide information for induction of respiratory cycle time of breathing of said examinee according to a period of said blood pressure fluctuations;
obtaining a parameter value related to intensity of said heartbeat waveform;
determining occurrence or non-occurrence of a problem in autonomic activity in an autonomic nervous system in said examinee according to said parameter value; and
outputting a determination result of said determining step.

12. A non-transitory computer readable medium for storing a computer-executable program enabling execution of computer instructions to perform operations for diagnosis support, said operations comprising:
obtaining information of a heartbeat waveform of an examinee;
extracting blood pressure fluctuations based on changes of blood pressure of said examinee from said heartbeat waveform;
outputting guide information for induction of respiratory cycle time of breathing of said examinee according to a period of said blood pressure fluctuations;
obtaining a parameter value related to intensity of said heartbeat waveform;
determining occurrence or non-occurrence of a problem in autonomic activity in an autonomic nervous system in said examinee according to said parameter value; and
outputting a determination result of said determining.
